(19) United States
(12) Patent Application Publication
(10) Pub. No.: US 2023/0034167 A1
(43) Pub. Date: Feb. 2, 2023

(54) MULTISENSORIAL INTELLIGENCE FOOTWEAR

(71) Applicant: Microsoft Technology Licensing, LLC, Redmond, WA (US)

(72) Inventors: Evelina BARHUDARIAN, Renton, WA (US); Alemayehu SEYED, Mercer Island, WA (US)

(21) Appl. No.: 17/472,062

(22) Filed: Sep. 10, 2021

Related U.S. Application Data
(60) Provisional application No. 63/226,501, filed on Jul. 28, 2021.

(51) Int. Cl. G01C 21/20; G01S 19/01; A43B 3/00; A61B 5/11; A61B 5/00

(52) U.S. Cl. CPC G01C 21/20; G01S 19/01; A43B 3/0005; A61B 5/112; A61B 5/6807; A61B 2503/10; H02J 7/35

(57) ABSTRACT

An intelligent shoe comprises one or more sensorial devices configured to generate sensorial signals to instruct a wearer to walk along a pedestrian path. The intelligent shoe is configured to cause a current location of the wearer to be determined and to obtain a pedestrian path between the current location and the destination location. The intelligent shoe then causes the one or more sensorial devices to generate sensorial signals, instructing the wearer to move along the pedestrian path.

### CROSS-REFERENCE TO RELATED APPLICATION

This application claims the benefit of U.S. Provisional Application No. 63/226,501, filed Jul. 28, 2021, the entire contents of which are herein incorporated by reference.

### BACKGROUND

There are many benefits of walking, including (but not limited to) physical and mental health benefits. Many people enjoy walking outdoors in nature and/or in town. However, it can be difficult to navigate strange areas and/or in the dark. Further, for blind or visually impaired (BVI), and/or hearing impaired individuals, it is even more difficult to navigate outdoor, let alone a strange area.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter.

The embodiments described herein are related to a wearable (such as an intelligent shoe), including one or more sensorial devices configured to use sensorial signals to help a wearer to navigate to different destination locations. Such a wearable or an intelligent shoe can help a wearer with impaired vision or hearing to navigate outdoor and/or help any wearer to navigate any place at any time.

The intelligent shoe is configured to cause a current location of a wearer to be determined. The intelligent shoe is also configured to obtain a pedestrian path between the current location and a destination location. In response to obtaining the pedestrian path, the intelligent shoe is configured to instruct the wearer to walk along the pedestrian path from the current location to the destination location. In some embodiments, the one or more sensorial devices are configured to instruct the wearer to move forward, turn around, turn left, and/or turn right.

In some embodiments, the intelligence shoe is further configured to communicate with a server and receive the pedestrian path from the server. The server is configured to generate the pedestrian path from the current location to the destination location and send the pedestrian path to the intelligent shoe. In some embodiments, the intelligent shoe is further configured to pair with a mobile device configured to communicate with a server and receive the pedestrian path from the server via the mobile device.

As such, the embodiments described herein are also related to a server computing system configured to communicate with a plurality of intelligent shoes. In some embodiments, the server computing system is configured to receive geolocation data from a plurality of intelligent shoes and generate a heatmap indicating historical frequencies of visits of different areas based on the geolocation data received from the plurality of intelligent shoes. The server computing system is also configured to receive a navigation request containing at least a current location of a wearer of the intelligent shoe among the plurality of intelligent shoes. In response to receiving the navigation request, the server computing system is configured to generate a pedestrian path from a current location of the wearer to a destination location based on the heatmap indicating the historical frequencies of visits of different areas. The pedestrian path is then caused to be received by the intelligent shoe, causing the intelligent shoe to generate sensorial signals to instruct the wearer to walk along the pedestrian path.

The principles described herein are also related to a mobile device configured to pair with an intelligent shoe having one or more sensorial devices. The mobile device is configured to determine a current location of itself and receive a user input, indicating a navigation request. The mobile device then obtains a pedestrian path from the current location to a destination location associated with the navigation request. The mobile device then sends the pedestrian path to the intelligent shoe, causing the intelligent shoe to generate sensorial signals to instruct the wearer to walk along the pedestrian path.

Additional features and advantages will be set forth in the description which follows, and in part will be obvious from the description or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not, therefore, to be considered to be limiting in scope, embodiments will be described and explained with additional specificity and details through the use of the accompanying drawings in which:

FIG. 1 illustrates an example system including one or more intelligent shoes, one or more mobile devices, and a server computing system that implement the principles described herein;

FIG. 2 illustrates an example architecture of an intelligent shoe corresponding to the one or more intelligent shoes of FIG. 1;

FIG. 3 illustrates example sensorial devices that may be implemented in the intelligent shoe of FIG. 2;

FIG. 4 illustrates an example power source that may be implemented in the intelligent shoe of FIG. 2;

FIG. 5 illustrates example sensing devices that may be implemented in the intelligent shoe of FIG. 2;

FIG. 6 illustrates an example embodiment of smart fabric that may be implemented as sensing devices of FIG. 5;

FIG. 7 illustrates an example architecture of a server computing system corresponding to the server computing system of FIG. 1;

FIG. 8 illustrates an example architecture of a mobile device corresponding to the mobile device of FIG. 1;

FIG. 9A illustrates an example map showing two pedestrian paths that have been traversed by a same wearer of an intelligent shoe or different wearers of different intelligent shoes;

FIG. 9B illustrates an example map showing several pedestrian paths that have been traversed by a same wearer of an intelligent shoe or different wearers of different intelligent shoes;

FIG. 9C illustrates an example heatmap generated based on the pedestrian paths of FIG. 9B;

FIG. 10 illustrates a flowchart of an example method for generating sensorial signals to instruct a wearer to move along the pedestrian path;

FIG. 11 illustrates a flowchart of an example method for accumulating geolocation data from a plurality of intelligent shoes and generating pedestrian path based on the accumulated geolocation data;

FIG. 12 illustrates a flowchart of an example method for connecting an intelligent shoe with a server computing system and causing the intelligent shoe to receive a pedestrian path generated by the server computing system;

FIG. 13 illustrates a flowchart of an example method for accumulating personal sensing data from at least one intelligent shoe and identify patterns associated with the wearer for improving the wearer's athletic performance and/or inferring the wearer's health conditions; and

FIG. 14 illustrates an example computing system in which the principles described herein may be employed.

### DETAILED DESCRIPTION

The principles described herein are related to a wearable (e.g., an intelligent shoe) having multisensorial devices configured to generate sensorial signals to help a wearer to navigate to different destinations. Such a wearable or an intelligent shoe can help a wearer with impaired vision or hearing to navigate outdoor and/or help any wearer to navigate any place at any time. Even though the descriptions herein are primarily directed to an intelligent shoe, similar principles described herein may also be implemented on other wearables, such as an intelligent shirt, intelligent belt, intelligent backpack, intelligent hat, intelligent watch, intelligent pants for the same or similar purposes.

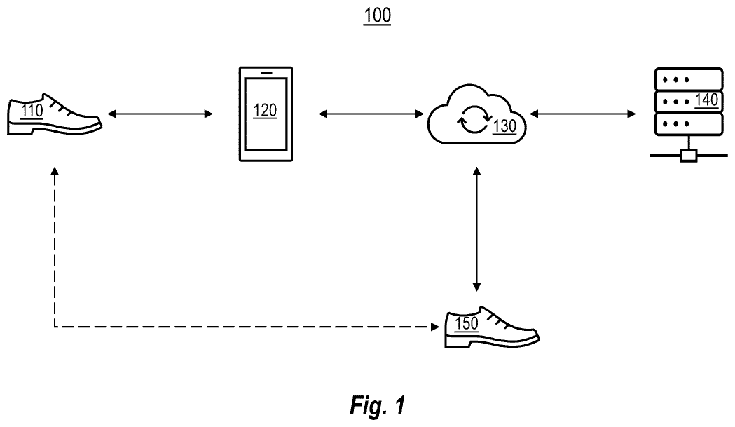

FIG. 1 illustrates an example of a system 100 that implements the principles described herein. As illustrated, the system 100 includes one or more intelligent shoes 110 and 150. In some embodiments, some intelligent shoes (e.g., intelligent shoe 150) is configured to be directly connected to a network 130 (e.g., via a wide area network, such as 3G, 4G, 5G wireless connection) and communicate with a server computing system 140 via the network 130. In some embodiments, some of the intelligent shoes (e.g., intelligent shoe 110) are configured to be connected to a mobile device 120 (e.g., via Bluetooth low energy connection), which is connected to a network 130 and configured to communicate with the server computing system 140. As such, the intelligent shoe 110 is configured to communicate with server computing system 140 via the mobile device 120.

In some embodiments, the intelligent shoe (e.g., intelligent shoe 150) is further configured to send its current location and/or a navigation request to a server (e.g., server computing system 140), causing the server (e.g., server computing system 140) to generate the pedestrian path from the current location to a destination location based on the navigation request. The intelligent shoe (e.g., intelligent shoe 150) then receives the pedestrian path from the server (e.g., server computing system 140).

In some embodiments, some intelligent shoes (e.g., intelligent shoe 110) are configured to be paired with a mobile device (e.g., mobile device 120) that is configured to communicate with a server (e.g., server computing system 140). Since both the mobile device and the intelligent shoe are carried or worn by the wearer, the current location of the intelligent shoe and the current location of the mobile device are substantially the same. In some embodiments, the current location of the intelligent shoe (or the mobile device) and a navigation request are sent to the server via the mobile device (e.g., mobile device 120). The server computing system 140 then generates and sends a pedestrian path to the mobile device 120, which in turn passes the pedestrian path to the intelligent shoe (e.g., intelligent shoe 110).

In some embodiments, the nearby intelligent shoes 110, 150 are configured to communicate with each other directly (e.g., via Bluetooth low energy) or indirectly (e.g., via a wide area network, such as 3G, 4G, 5G wireless network, or via mobile devices paired therewith). For example, in some embodiments, a first intelligent shoe may be configured to follow a second intelligent shoe or to navigate to a location of the second intelligent shoe, where the wearer of the first intelligent shoe and the wearer of the second intelligent shoe may be members of a same group in an organized outing event. As another example, in some embodiments, the intelligent shoes may also be configured to train athletes of team sports. Each of the athletes in a training session is wearing an intelligent shoe. The intelligent shoes are configured to detect the nearby wearers' motions, including (but not limited to) direction, speed, acceleration, and instruct its own wearer to move in a particular direction.

In some embodiments, only one of a pair of shoes is an intelligent shoe. In some embodiments, both shoes are intelligent shoes, and the pair of intelligent shoes are configured to be paired with each other and exchange data between each other. In some embodiments, each of the pair of intelligent shoes is configured to communicate with the server computing system 140 or the mobile device 120 separately. In some embodiments, only one of the pair of intelligent shoes is configured to communicate with the mobile device 120 and/or the server computing system 140. In some embodiments, the pair of intelligent shoes are configured to take turns to communicate with the mobile device 120 and/or the server computing system 140 based on the power usage, such that the power level of each intelligent shoe is kept substantially even.

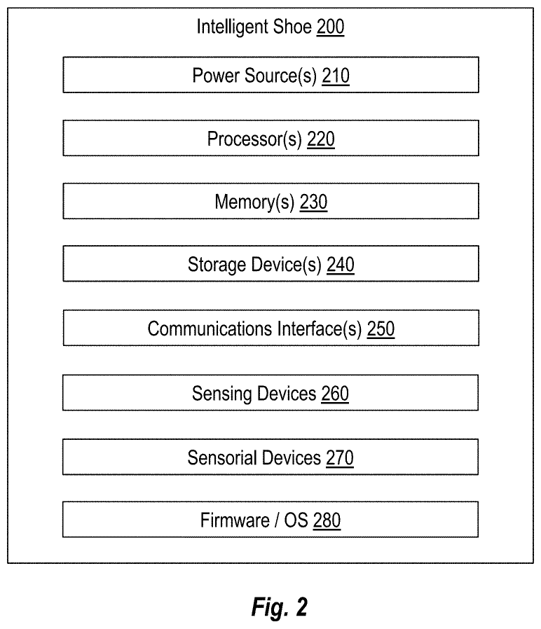

FIG. 2 illustrates an example architecture of an intelligent shoe 200 that corresponds to the intelligent shoe 110 or 150 of FIG. 1. As illustrated, the intelligent shoe 200 includes one or more power sources 210, one or more processors 220, one or more memories 230, one or more storage devices 240, one or more communications interfaces 250 (e.g., Bluetooth low energy interface, a Wi-Fi interface, a 3G, 4G, and/or 5G communication interface), and/or one or more sensorial devices 270. In some embodiments, the power source 210, the processor(s) 220, the memory(s) 230, the storage device(s) 240, and/or the communication interface(s) 250 are packaged into a dongle that is configured to be attached or detached from the intelligent shoe 200. In some embodiments, all these components are integrated into the intelligent shoe 200 and not intended to be removable.

Further, in some embodiments, firmware or an operating system 280 is stored in the one or more storage devices 240 and loaded in the one or more memories 230. In some embodiments, the firmware or the operating system 280 is configured to process sensing data received from the sensing devices 260 and transmit the sensing data to a mobile device (e.g., mobile device 120) or a server computing system (e.g., server computing system 140) via the one or more communication interfaces 250. In some embodiments, the firmware or the operating system 280 is further configured to allow the intelligent shoe 200 to receive data from a mobile device (e.g., mobile device 120) or a server computing system (e.g., server computing system 140) via the one or more communication interfaces 250. In some embodiments, the data received from the mobile device or the server computing system include (but are not limited to) a pedestrian path between the current location of the wearer and a destination location.

The firmware or operating system 280 is further configured to cause the one or more sensorial devices 270 to generate sensorial signals based on a pedestrian path (which may be received from a mobile device or a server, or be generated by the processor(s) 220 of the intelligent shoe 200). The sensorial signals are configured to instruct a wearer of the intelligent shoe 200 to walk along the pedestrian path from the current location to the destination location. In some embodiments, the one or more sensorial devices are configured to instruct the wearer to move forward, turn around, turn left, turn right, and/or stop.

In some embodiments, the intelligent shoe 200 further includes one or more sensing devices 260 configured to detect environmental data and/or personal data. In some embodiments, the intelligent shoe 200 is further configured to record geolocation data associated with the intelligent shoe in the one or more storage devices 240. In some embodiments, the intelligent shoe 200 is further configured to record environmental sensing data generated by the one or more sensing devices 260 in the one or more storage devices 240 relationally with the geolocation data. In some embodiments, the intelligent shoe 200 is further configured to record personal sensing data generated by the one or more sensing devices 260 in the one or more storage devices 240. In some embodiments, the intelligent shoe 200 is configured to process the geolocation data, the environmental data, and/or the personal data by itself to identify patterns. In some embodiments, the intelligent shoe 200 is configured to send such data to a mobile device via a personal area network (e.g., a Bluetooth low energy network). The mobile device is then configured to process such data to identify patterns. In some embodiments, the intelligent shoe 200 is configured to send such data to a server computing system directly or indirectly via a mobile device. The server computing system is then configured to process such data received from different intelligent shoes to identify patterns. In some embodiments, the server computing system is configured to use different machine learning techniques to analyze the data generated by different intelligent shoes to train one or more artificial intelligence (AI) models, and the trained AI models are then deployed to the mobile devices or the intelligent shoes to provide faster results to wearers.

For example, based on the personal data generated by the intelligent shoes, one or more AI models can be trained to identify abnormalities of the gait of a particular wearer. Such AI models can be deployed onto an intelligent shoe, such that the intelligent shoe is configured to detect abnormalities of the gait of a wearer and potentially help the wearer to correct the abnormalities. As another example, one or more AI models can also be trained to help an athlete to improve performance in a particular sport.

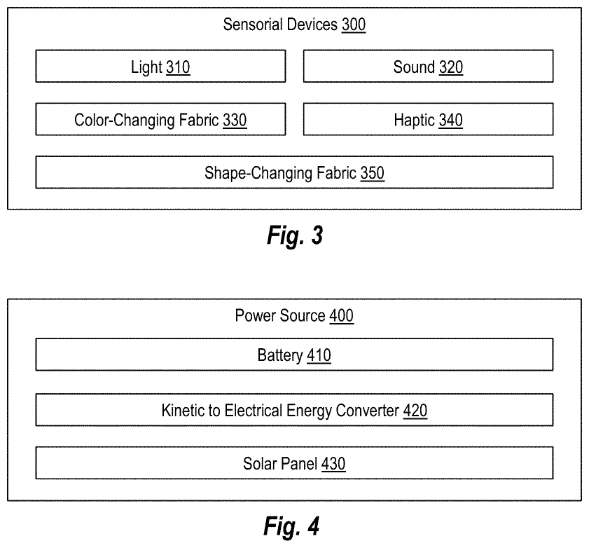

As briefly described with respect to FIG. 2, the intelligent shoe 200 includes one or more sensorial devices 270. FIG. 3 further illustrates example sensorial devices 300, which include (but are not limited to) one or more light-emitting devices 310 configured to emit light, one or more sound devices 320 configured to generate sound, one or more pieces of color-changing fabric 330, one or more haptic devices 340, and/or one or more pieces shape-changing fabric 350. In some embodiments, the light-emitting device(s) 310 includes one or more LED lights configured to flash at a pattern to instruct the wearer to walk in different directions. In some embodiments, the sound device(s) 320 includes one or more speakers configured to generate voice instructions to instruct the wearer to move in different directions. In some embodiments, the one or more pieces of color-changing fabric 330 are configured to change colors when different currents or voltages are applied to the fabric, sending different messages to the wearer. In some embodiments, the one or more haptic devices 340 are configured to provide haptic feedback to the wearer (such as different types of vibrations at different locations of the intelligent shoe 200) to send different messages to the wearer. In some embodiments, the one or more pieces of shape-changing fabric 350 are configured to change shape when different currents or voltages are applied to the fabric, sending different messages to the wearer.

Further, as briefly described with respect to FIG. 2, the intelligent shoe 200 also includes one or more power sources 210 configured to power the one or more processors 220, the one or more memories 230, and/or the one or more sensorial devices 270, etc. FIG. 4 illustrates an example embodiment of a power source 400, which corresponds to the power source 210 of FIG. 2. As illustrated in FIG. 4, in some embodiments, the power source 400 includes a battery 410. In some embodiments, the battery 410 is a disposable battery. In some embodiments, the battery 410 is a rechargeable battery. In some embodiments, the power source 400 further includes a kinetic to electrical energy converter 420 configured to convert kinetic energy of wearer of the intelligent shoe to electrical energy, and a rechargeable battery 410 configured to receive and store the electrical energy generated by the kinetic to electrical energy converter 420. In some embodiments, the power source 400 includes a solar panel 430 configured to convert ambient light to electrical energy, and a rechargeable battery 410 configured to receive and store the electrical energy generated by the solar panel 430.

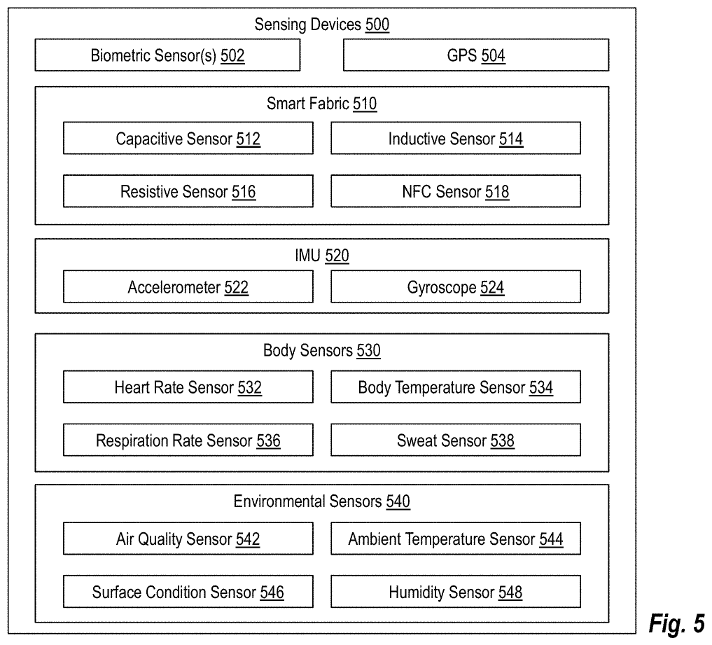

As illustrated in FIG. 2, in some embodiments, the intelligent shoe 200 may further include one or more sensing devices. FIG. 5 illustrates examples of one or more sensing devices 500 that may be implemented in the intelligent shoe 200, which corresponds to the one or more sensing devices 260 of FIG. 2. As illustrated in FIG. 5, in some embodiments, the one or more sensing devices 500 include one or more biometric sensors 502 configured to receive biometric data from a wearer (which may be used for securely authenticating the wearer) and/or a global positioning system (GPS) 504 configured to obtain geolocation data of the intelligent shoe.

In some embodiments, the one or more sensing devices 500 further include one or more pieces of smart fabric 510, each of which has an array of at least one of (1) one or more capacitive sensors 512, (2) one or more resistive sensors 516, (3) one or more inductive sensors 514, and/or (4) one or more NFC sensors 518.

In some embodiments, the one or more sensing devices 500 further include an inertial measurement unit (IMU) 520. In some embodiments, the IMU 520 includes an accelerometer 522 and a gyroscope 524. The IMU 520 is configured to detect foot motions of a wearer of an intelligent shoe, including (but not limited to) speed, direction, and/or acceleration of the foot of the wearer. In some embodiments, the data generated by the IMU 520 can be used to extract features associated with the gait of the wearer and/or detect abnormalities of the gait of the wearer.

In some embodiments, the one or more sensing devices 500 further include one or more body sensors 530 configured to detect body conditions. In some embodiments, the one or more body sensors 530 include (but are not limited to) a heart rate sensor 532, a body temperature sensor 534, a respiration rate sensor 536, and/or a sweat sensor 538. The data generated by the body sensors 530 can be used to infer different health conditions of a wearer of an intelligent shoe.

In some embodiments, the one or more sensing devices 500 further includes one or more environmental sensors 540 configured to detect environmental conditions. In some embodiments, the environmental sensors 540 include (but are not limited to) an air quality sensor 542, an ambient temperature sensor 544, a surface condition sensor 546, and/or a humidity sensor 548. In some embodiments, the data gathered by the environmental sensors 540 can be stored relationally with geolocation data gathered by the GPS 504, which can then be used to generate different heatmaps indicating environmental conditions in different areas.

As illustrated in FIG. 5, in some embodiments, the one or more sensing devices 500 may include smart fabric 510. The smart fabric may be implemented at any part of the shoes, such as (but not limited to) a sole of the shoe, a tongue of the shoe, a heal of the shoe, a toe cap of the shoe.

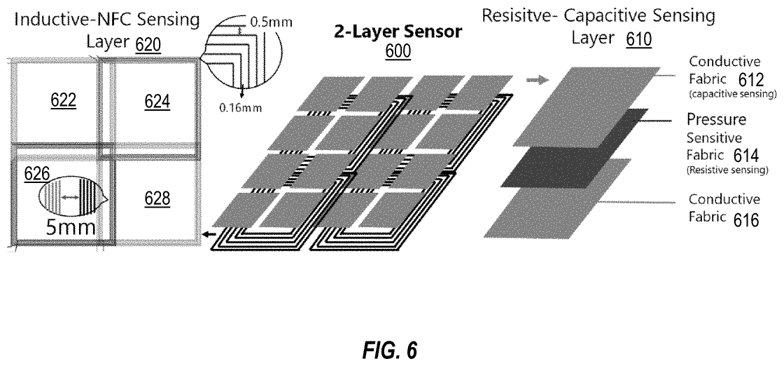

FIG. 6 illustrates an example piece of smart fabric 600 that corresponds to the smart fabric 510 of FIG. 5. As illustrated in FIG. 6, the piece of smart fabric 600 includes (1) a first layer 610 (also referred to as the “resistive-capacitive sensing layer”) having multiple resistive sensors and multiple capacitive sensors, and (2) a second layer 620 (also referred to as the “inductive-NFC sensing layer”) having multiple inductive sensors and multiple NFC sensors. Each of the multiple resistive sensors corresponds to each of the multiple NFC sensors, respectively. Each of the multiple inductive sensors corresponds to each of the multiple NFC sensors, respectively.

In some embodiments, in the first layer 610, each of the multiple capacitive sensors includes a first piece of conductive fabric 612 and a second piece of conductive fabric 616. In some embodiments, the conductive fabric 612, 616 includes non-metallic conductive fabric, such as a conductive polymer. Each of the multiple resistive sensors includes a piece of pressure sensing fabric 614, such as Velostat. In some embodiments, each piece of pressure sensing fabric 614 of the resistive sensor is sandwiched between the first piece of conductive fabric 612 and the second piece of conductive fabric 616 of the corresponding capacitive sensor. Each piece of the conductive fabric 612, 616, and each piece of pressure sensing fabric 614 serves as an electrode. The rows and columns of electrodes 612, 616 of the capacitive sensors are electrically separated, while the rows and columns of electrodes 614 of the resistive sensors are electrically connected. As illustrated, the resistive sensors and the capacitive sensors are arranged in a grid of 4×4, though the invention is not limited to this structure. For example, a greater number of capacitive sensors and/or resistive sensors (e.g., a grid of 4×8, 8×8, 16×16, 32×32, 64×64, etc.) may also be implemented.

The second layer 620 includes multiple coils 622, 624, 626, 628 configured to act as both inductor coils for the multiple inductive sensors and sensor coils for the multiple NFC sensors. In some embodiments, every two adjacent coils among the plurality of coils overlap each other. Because an operation may become unreliable when the inductance of the coils is below 4 uH, it is advantageous to implement the coils 622, 624, 626, 628 to have an inductance of at least 4 uH, and about 5 or more traces are preferred. As illustrated, four coils are arranged in a grid of 2×2, and each coil is rectangular-shaped or square-shaped and has about 5 traces, though the invention is not limited to this structure. For example, a different shape of coils (e.g., circular-shaped) or a greater number of coils (e.g., a grid of 2×4, 4×4, 8×8, 16×16, 32×32, 64×64, etc.) may also be implemented.

Each of the multiple capacitive sensors is configured to sense touch input. Each of the multiple resistive sensors is configured to sense pressure based on the change in the resistance of a pressure-sensitive material (such as piezo-resistive material) when it is pressed or deformed. Thus, the resistive sensors are also configured to sense touch input.

Each of the multiple NFC sensors uses alternating electromagnetic fields for receiving and transmitting data. When an NFC sensor is triggered by an electromagnetic interrogation signal from a nearby antenna coil, it transmits its data to the sensor coil. In some embodiments, each NFC sensor includes a coil that is laid out in a particular manner, such that the NFC sensor not only can detect tags, but also can function as an inductive sensor. Note, although the smart fabric 600 illustrated in FIG. 6 includes capacitive sensors and inductive sensors, the inductive sensors are not necessarily required. In some embodiments, the smart fabric 600 only includes a layer of capacitive sensors and resistive sensors. Further, it is also not necessary that a grid of 2×2 capacitive sensors corresponds to one NFC sensor.

As briefly described above, in some embodiments, the intelligent shoe 110, 150, 200 is also configured to directly or indirectly communicate with a server computing system 140. In particular, in some embodiments, the intelligent shoe 110 is configured to transmit geolocation data, personal data, and/or environmental data to the server computing system 140, and the server computing system 140 is configured to process the data received from different intelligent shoes to identify patterns. As such, the principles described herein are also related to a server computing system (e.g., server computing system 140).

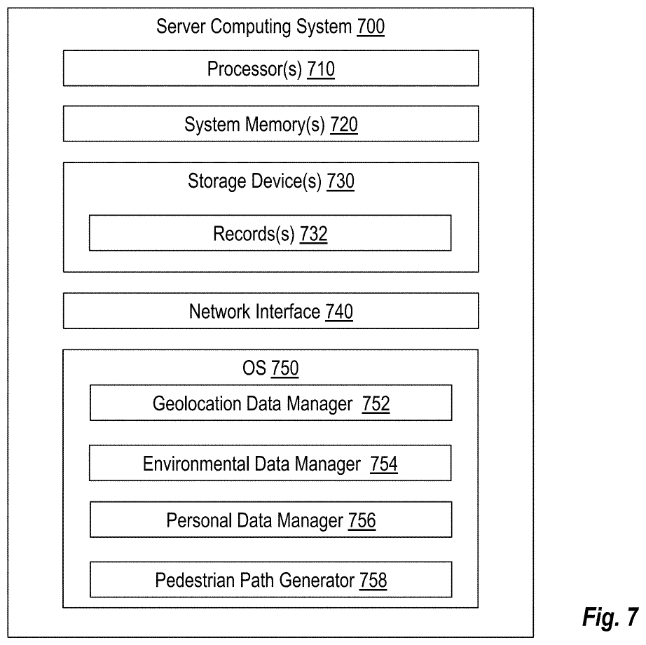

FIG. 7 illustrates an example architecture of a server computing system 700, which corresponds to server computing system 140 of FIG. 1. As illustrated in FIG. 7, the server computing system 700 includes one or more processors 710, one or more system memories 720, one or more storage devices 730, and a network interface 740. The one or more storage devices 730 are configured to receive the geolocation data, personal data, and/or environmental data generated from the plurality of intelligent shoes via the network interface 740 and store the received geolocation data as a plurality of records 732.

In some embodiments, an operating system 750 is installed in the one or more storage devices 730 and loaded in the one or more system memories 720. In some embodiments, a location data manager 752, an environmental data manager 754, and/or a personal data manager 756 are installed in the operating system 750. The location data manager 752 is configured to manage and process the geolocation data received from the plurality of intelligent shoes and generate a heatmap indicating historical frequencies of visits of different areas based on the received geolocation data.

In some embodiments, the server computing system 700 is also configured to receive environmental data (e.g., environmental data generated by the one or more environmental sensors 540) from the plurality of intelligent shoes via the network interface 740 and store the environmental data relationally with the geolocation data as one or more records 732 in the storage devices 730. The environmental data manager 754 is configured to manage and process the environmental data received from the plurality of intelligent shoes. In some embodiments, the environmental data manager 754 is also configured to generate one or more heatmaps indicating environmental conditions in different areas.

The personal data manager 756 is configured to accumulate and manage personal data generated by the smart fabric 510, the body sensor(s) 530, and/or IMU 520. In some embodiments, the personal data manager 756 is configured to manage and process the personal data received from the plurality of intelligent shoes. Such data may be analyzed to achieve different goals, such as (but not limited to) inferring the wearer's health conditions and/or improving the wearer's athletic performance. For example, in some embodiments, the personal data manager 756 is configured to analyze the personal data associated with gait of the at least one wearer to identify abnormalities.

In some embodiments, a pedestrian path generator 758 is also installed in the operating system 750 of the server computing system 700. In some embodiments, the server computing system 700 is configured to receive a navigation request containing a current location and a destination location from a wearer of an intelligent shoe (e.g., intelligent shoe 110, 200) among the plurality of intelligent shoes. In response to receiving the navigation request, the pedestrian path generator 758 is configured to generate a pedestrian path from the current location to the destination location. In some embodiments, the server computing system 700 is further configured to send the pedestrian path to the intelligent shoe, causing one or more sensorial devices of the intelligent shoe to generate sensorial signals, instructing the wearer of the intelligent shoe to walk along the pedestrian path.

In some embodiments, the pedestrian path is generated based on the heatmap indicating frequencies of visits of different areas and/or the heatmap indicating environmental conditions of different areas. In some embodiments, the pedestrian path is generated based on most traveled areas or least traveled areas during a particular time period in the heatmap. In some embodiments, the pedestrian path is generated based on the environmental data associated with areas between the current location and the destination location.

In some embodiments, the pedestrian path generator 758 is further configured to generate a random pedestrian path in response to receiving a navigation request from a wearer of an intelligent shoe among the plurality of intelligent shoes. In some embodiments, the navigation request may include one or more attributes of a destination location, and the pedestrian path generator 758 is configured to identify a destination location that satisfies the attributes requirement. For example, in some cases, the wearer may merely request a random path within a particular distance of walking, and the pedestrian path generator 758 is configured to generate a random path of the particular distance. In some embodiments, the pedestrian path generator 758 is configured to work with an existing map system, such as BING. For example, the wearer may request a popular restaurant within a particular distance of walking, and the pedestrian path generator 758 is configured to identify such a popular restaurant based on the existing map system (having restaurants locations) and the heatmap of frequency of visit during a particular time period (e.g., around noon, between 7 pm and 9 pm).

Further, as briefly described above, in some embodiments, the intelligent shoe 110, 150, 200 is also configured to be paired with a mobile device, which in turn passes the communication between the intelligent shoe and a server computing system. As such, the principles described herein are also related to a mobile device (e.g., mobile device 120). The mobile device is configured to be paired with an intelligent shoe (e.g., intelligent shoe 110) having one or more sensorial devices and communicate with a server computing system.

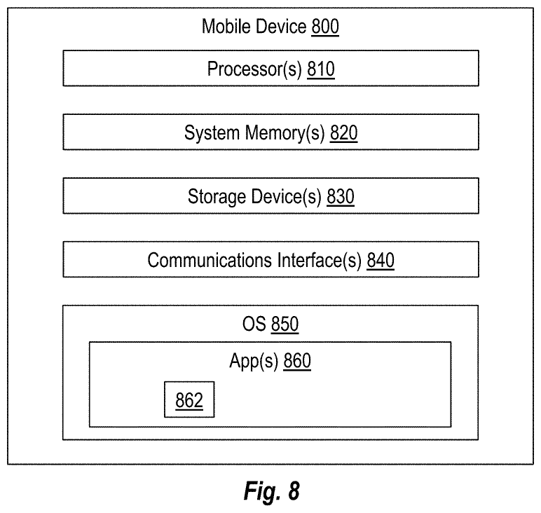

FIG. 8 illustrates an example architecture of a mobile device 800, which corresponds to mobile device 120 of FIG. 1. As illustrated in FIG. 8, the mobile device 800 includes one or more processors 810, one or more system memories 820, one or more storage devices 830, and one or more communication interfaces 840 (e.g., Bluetooth low energy interface, a Wi-Fi interface, a 3G, 4G, and/or 5G communication interface). In some embodiments, an operating system 850 is installed on the one or more storage devices 830 and loaded in the one or more system memories 820. In some embodiments, one or more applications 860 are installed in the operating system 850. In some embodiments, the one or more applications 860 includes a user agent application 862 configured to communicate with an intelligent shoe (e.g., intelligent shoe 110) and/or a server (e.g., server computing system 140).

In some embodiments, the user agent application 862 is also configured to determine a current location of the mobile device 800 and receive a user input, indicating a navigation request. In some embodiments, the mobile device 800 is configured to accumulate location data of the wearer, and based on the location data of the wearer to generate a pedestrian path. In some embodiments, the mobile device is configured to obtain a pedestrian path from a server. The pedestrian path generated by the mobile device 800 or server is then sent to the intelligent shoe, causing the one or more sensorial devices of the intelligent shoe to generate sensorial signals to instruct a wearer of the intelligent shoe to walk along the pedestrian path.

In some embodiments, the wearer can request a random pedestrian path via the user agent application 862. For example, in some embodiments, the wearer can enter a distance that the wearer would like to walk (e.g., one mile, three miles, etc.) via a user interface of the user agent application 862. Receiving the user input, in some embodiments, the mobile device 800 can generate a random pedestrian path by itself. Alternatively, the user agent application 862 is configured to pass the user input to a server computing system (e.g., server computing system 140, 700), which in turn generates a random pedestrian path with the distance entered by the wearer. In some embodiments, the wearer can also indicate whether the wearer prefers the most traveled path or least traveled path and/or the condition of the road (e.g., flat, particular vertical elevation change, etc.). In some embodiments, one wearer may be configured to share their path with another wearer. In some embodiments, wearers can choose to share their historical paths with other wearers publicly or anonymously, and other wearers can select one of those shared paths.

In some embodiments, the mobile device 800 is configured to display the heatmap generated by the server computing system 700 and/or the pedestrian path to the destination location or the random pedestrian path. In some embodiments, the mobile device 800 is also configured to display a pedestrian path the wearer has traversed through.

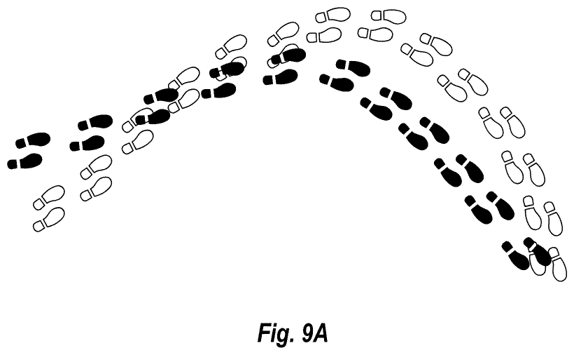

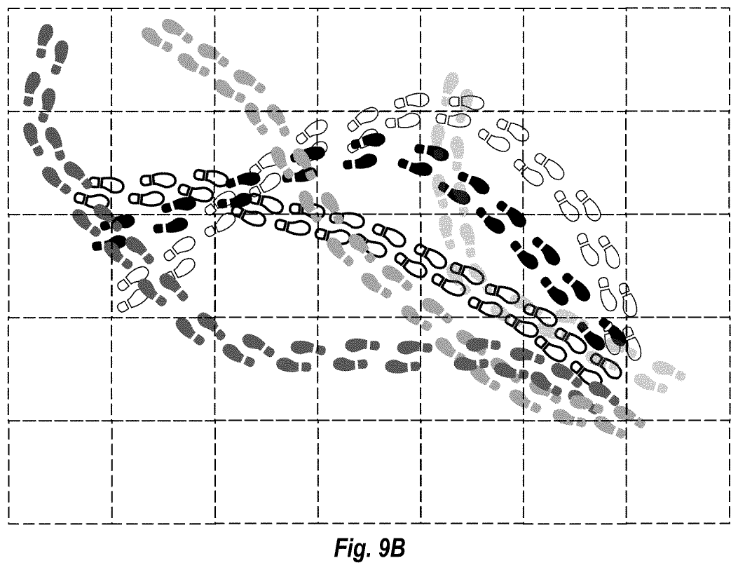

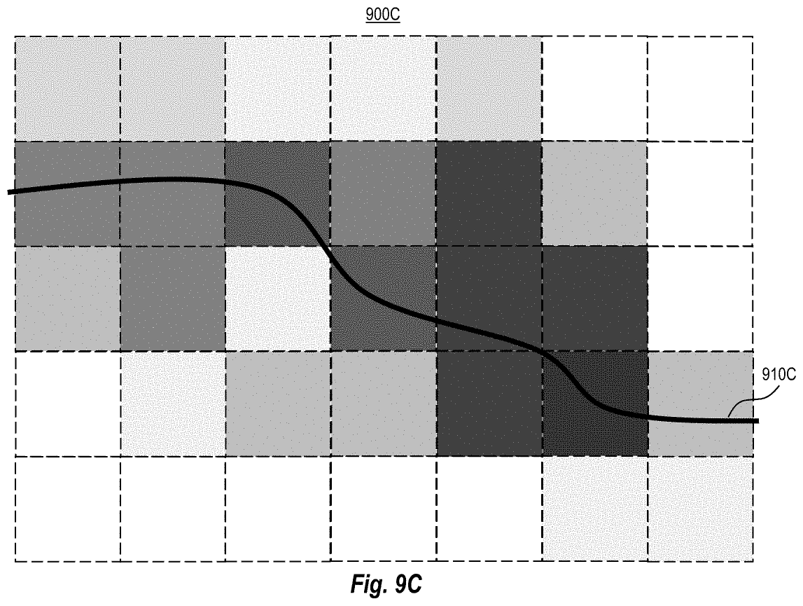

FIG. 9A illustrates a map showing two pedestrian paths that have been traversed by a same wearer of an intelligent shoe or different wearers of different intelligent shoes. FIG. 9B illustrates a map showing additional pedestrian paths that have been traversed by wearer(s). The different pedestrian paths of many different wearers may then be processed to generate a heatmap. In some embodiments, the more frequently stepped-over areas are colored in a darker color, and the less frequently stepped-over areas are colored in a lighter color. FIG. 9C illustrates an example heatmap 900C that is generated based on the pedestrian paths shown in FIG. 9B. As illustrated in FIGS. 9B and 9C, the areas that include more footsteps in FIG. 9B are colored in darker colors, and the areas that contain fewer footsteps in FIG. 9B are colored in lighter colors. In some embodiments, the server computing system 700 or the mobile device 800 is configured to identify pedestrian paths between two given locations based on the heatmap. For example, line 910C shown in FIG. 9C may be identified as a pedestrian path based on the heatmap 900C.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

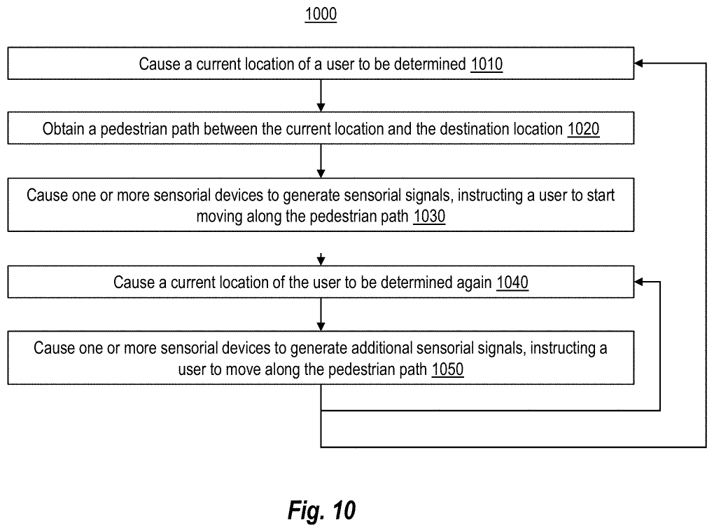

FIG. 10 illustrates a flowchart of an example method 1000 for generating sensorial signals to instruct a wearer to walk along a pedestrian path., which may be performed by an intelligent shoe (e.g., intelligent shoe 110, 150, 200). The method 1000 includes causing a current location of a wearer to be determined (act 1010). In some embodiments, the intelligent shoe includes a GPS, and the determination of the current location of the wearer is performed by itself. In some embodiments, the intelligent shoe is paired with a mobile device of the wearer, and the determination of the current location of the wearer is performed by the mobile device.

The method 1000 also includes obtaining a pedestrian path between the current location and a destination location (act 1020). In some embodiments, the destination location may be entered by the wearer directly to the intelligent shoe (such as a voice command, a textual input via a touch input interface of the intelligent shoe). In some embodiments, the destination location is input into a mobile device that is paired with the intelligent shoe. In some embodiments, the destination location may be a specified location that is input by the wearer. In some embodiments, the destination location may be randomly generated. In some embodiments, the randomly generated destination location may be required to meet certain criteria set by the wearer. For example, in some embodiments, the wearer can set a distance of walking, and the intelligent shoe is configured to generate a random pedestrian path based on the wearer's past walking history.

Based on the destination location, a pedestrian path may be generated. In some embodiments, the pedestrian path is generated based on the wearer's past walking history and generated by the intelligent shoe itself or a mobile device paired with the intelligent shoe. In some embodiments, the pedestrian path is based on many different wearers' past walking history and generated by a server computing system. The server computing system is configured to send the path to the intelligent shoe directly or indirectly via a mobile device. In some embodiments, the wearer's or many different wearer's past walking histories can be used to generate a heatmap, indicating the areas that are reachable by walking, and the pedestrian path is generated based on the heatmap indicating the frequencies of visits.

Receiving the pedestrian path between the current location and the destination location (act 1020), the intelligent shoe is configured to cause one or more sensorial devices to generate sensorial signals, instructing a wearer to start moving along the pedestrian path (act 1030). The one or more sensorial devices may include (but are not limited to) one or more LED lights, one or more sound devices, one or more color-changing fabric, one or more haptic devices, and/or one or more pieces of shape-changing fabric. For example, the LED lights may be configured to generate different light patterns indicating different directions. As another example, the sound devices may be configured to generate voice commands instructing the wearer to walk in different directions. As another example, the haptic devices may be configured to create haptic feedback at different parts of the intelligent shoe to indicate different directions. As another example, the shape-changing fabric may be configured to change its shape to create a gentle squeeze at different parts of the intelligent shoe to indicate different directions.

Notably, when the wearer walks based on the instruction of the sensorial signals, the location of the wearer would change. The method 1000 also includes causing a current location of the wearer to be determined again (act 1040) and causing one or more sensorial devices to generate additional sensorial signals, instructing a wearer to move along the pedestrian path (act 1050). For example, when it is determined that the wearer is currently at an intersection, the intelligent shoe is configured to generate additional sensorial signals to instruct the wearer which direction to turn to. Act 1040 and act 1050 may repeat as many times as necessary until the wearer reaches the destination or until the wearer changes their destination. In some embodiments, the wearer's location is determined at a predetermined time interval (e.g., 2 seconds, 10 seconds), and a new set of sensorial signals is generated at the same frequency. In some embodiments, a new set of sensorial signals is generated only when the wearer is required to make turns or change directions.

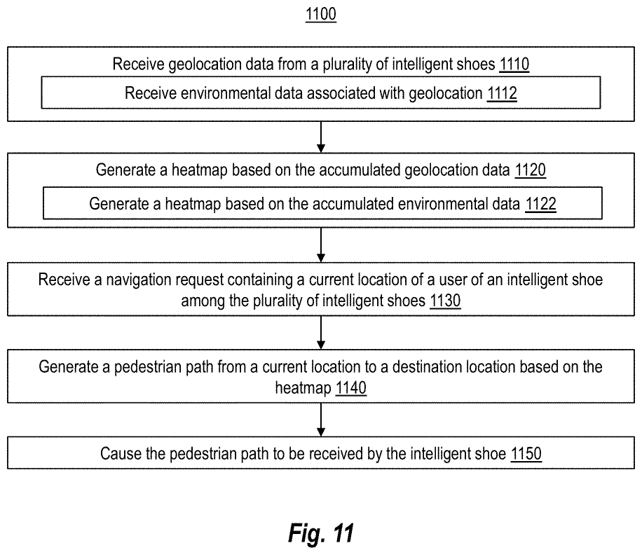

Principles described herein are also related to a server computing system configured to gather geolocation data from intelligent shoes and generate pedestrian paths based on the gathered geolocation data. FIG. 11 illustrates a flowchart of an example method 1100 for gathering geolocation data from intelligent shoes and generate pedestrian paths based on the gathered geolocation data, which may be performed by a server computing system (e.g., server computing system 140, 700). The method 1100 includes receiving geolocation data from a plurality of intelligent shoes (act 1110). In some embodiments, the server computing system is also configured to receive environmental data associated with geolocations and store the environmental data relationally with the geolocation data (act 1112). The method 1100 also includes generating a heatmap based on the accumulated geolocation data, reflecting the historical frequencies of visits of different areas (act 1120). For example, the most traveled areas of the heatmap may be colored in a darker and/or hotter color, and the least traveled areas of the heatmap may be colored in a lighter and/or colder color. In some embodiments, the server computing system is also configured to generate a heatmap based on the environmental data (act 1122). For example, in some embodiments, the heatmap may indicate the air qualities of different areas. As another example, in some embodiments, the heatmap may indicate the road conditions of different areas, such as (but not limited to) good condition, poor condition, flat, rocky, 5 degrees of uphill/downhill vertical angle, etc.

The method 1100 also includes receiving a navigation request containing at least a current location of a wearer of an intelligent shoe among the plurality of intelligent shoes (act 1130). In some embodiments, the navigation request is received directly from the intelligent shoe via a wide area network (e.g., 3G, 4G, 5G wireless network). In some embodiments, the navigation request is received from a mobile device that is paired with the intelligent shoe. The method 1100 also includes generating a pedestrian path from a current location to a destination location based on the heatmap that reflecting the frequency of visits and/or the heatmap that reflects the environmental condition (act 1140). Finally, the pedestrian path is caused to be received by the intelligent shoe (act 1150). Similarly, in some embodiments, the server computing system is configured to send the pedestrian path directly to the intelligent shoe via a wide area network (e.g., 3G, 4G, 5G wireless network). In some embodiments, the server computing system is configured to send the pedestrian path to the mobile device that is paired with the intelligent shoe, which in turn passes the pedestrian path to the intelligent shoe via a personal area network (e.g., a Bluetooth low energy network). Receiving the pedestrian path, the intelligent shoe is configured to cause its sensorial devices to generate sensorial signals, instructing the wearer to walk along the pedestrian path.

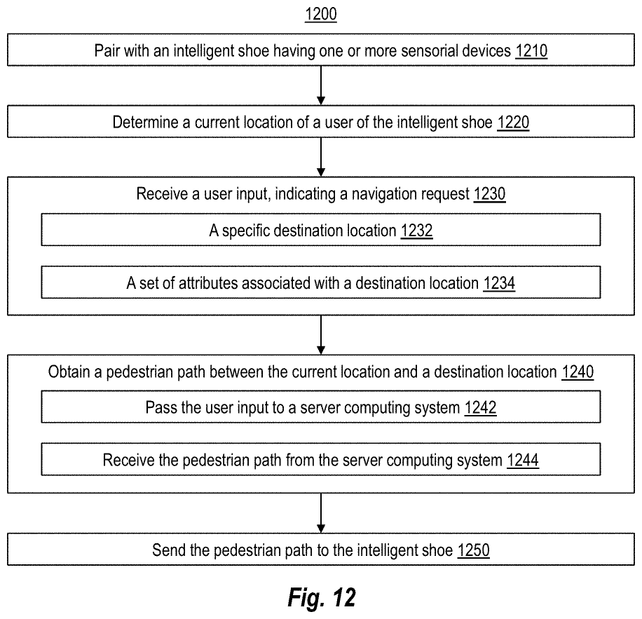

The principles described herein are also related to a mobile device (e.g., a mobile phone) configured to connect an intelligent shoe with a server computing system and to cause a pedestrian path to be received by the intelligent shoe. FIG. 12 illustrates a flowchart of an example method 1200 for causing a server computing system to generate a pedestrian path and causing the pedestrian path to be received by an intelligent shoe, which may be performed by a mobile device (e.g., mobile device 120, 800). The method 1200 includes pairing with an intelligent shoe having one or more sensorial devices (act 1210). The method 1200 further includes determining a current location of a wearer of the intelligent shoe (act 1220).

Notably, the mobile device and the intelligent shoe are both carried or worn by the wearer. Thus, the location of the wearer may be determined based on the location of the intelligent shoe or the location of the mobile device. In some embodiments, the intelligent shoe includes a GPS configured to generate geolocation data of itself, the geolocation data of the intelligent shoe is transmitted to the mobile device as the location of the wearer. In some embodiments, the mobile device includes a GPS configured to generate geolocation data of itself, the geolocation data of the mobile device is deemed as the location of the wearer. In some embodiments, when multiple sets of geolocation data are generated, the mobile device is configured to determine the reliability of each set of geolocation data and use the set of geolocation data that has the highest reliability. Alternatively, the mobile device may be configured to use an average value of the multiple sets of geolocation data.

The method 1200 also includes receiving a wearer input, indicating a navigation request (act 1230). In some embodiments, the wearer input may be entered by interacting with a touch screen or a keyboard of the mobile device. In some embodiments, the wearer input may be a voice command. In some embodiments, the wearer input includes a specific destination location (act 1232), such as (but not limited to) a particular restaurant, a particular store, a particular business office. In some embodiments, the wearer input includes a set of attributes associated with a destination location (act 1234). For example, in some cases, the wearer may indicate a popular restaurant within a specific distance of walking. As another example, the wearer may indicate “surprise me” or “walk a mile.” As another example, the wearer may indicate to follow another wearer. For example, in a group outing event, each member may be free to walk around in a large area but need to be regathered at a particular time of the day. Each of the members is wearing an intelligent shoe, and the location of a group leader may be set as the reference point, and the rest of the members may instruct their respective intelligent shoes to find and reach the group leader at a particular time.

Further, in response to receiving the navigation request, the mobile device is configured to obtain a pedestrian path between the current location and a destination location (act 1240). In some embodiments, the pedestrian path may be generated by the mobile device itself. In some embodiments, the mobile device is configured to pass the wearer input to a server computing system (act 1242), which in turn generates the pedestrian path and sends the pedestrian path to the mobile device. The mobile device then receives the pedestrian path from the server computing system (act 1244). After obtaining the pedestrian path (act 1240), the mobile device then sends the pedestrian path to the intelligent shoe (act 1250), causing the pedestrian path to generate sensorial signals to instruct the wearer to walk along the pedestrian path.

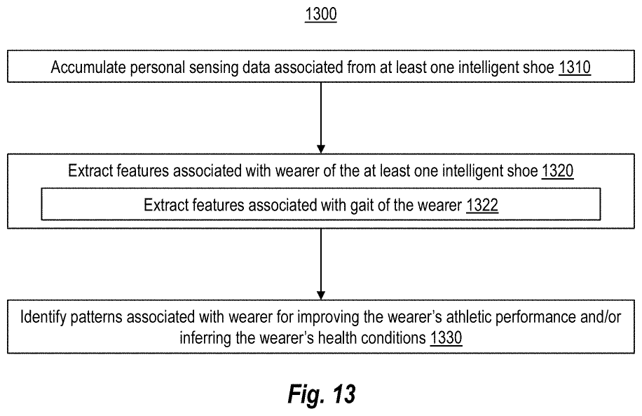

Further, as discussed above with respect to FIG. 5, in some embodiments, the intelligent shoe 200 may also include additional sensing devices, such as touch-sensitive smart fabric 510, IMU 520, and/or body sensors 530, which are configured to collect personal data of wearers. In some embodiments, these personal data are also gathered and analyzed (e.g., via different machine learning techniques) to identify patterns. These patterns can be used to improve the wearers' athletic performance and/or infer the wearers' health conditions. FIG. 13 illustrates a flowchart of an example method 1300 for gathering and analyzing sensing data from intelligent shoes, which may be performed by a server computing system (e.g., server computing system 140, 700). The method 1300 includes accumulating personal sensing data from at least one intelligent shoe (act 1310) and extract one or more features associated with at least one wearer of the at least one intelligent shoe (act 1320). In some embodiments, the one or more features associated with the wearer may be features associated with gait of the wearer (act 1322). Based on the features associated with the wearer, one or more patterns associated with the wearer may be identified for improving the wearer's athletic performance (act 1330). In some embodiments, the personal sensing data, such as (but not limited to) heart rate, respiratory rate, step speed, step length, step width, step height of the wearer, can also be used to infer the wearer's health conditions (act 1330).

It is advantageous to embed certain sensors in the shoes. For example, smart fabric, various body sensors, and/or IMU embedded in the intelligent shoe is likely to provide better measurement of a wearer's motion than a wrist band or a watch, and the data collected by the sensors in the intelligent shoe can be used to derive more accurate insights, such as (but not limited to) pro-athlete performance and/or biometrics.

In some embodiments, the smart fabric, sensing devices, and/or sensorial devices 500 shown in FIG. 5 may also be implemented in other wearables, such as (but not limited to) smart shirts/pants/uniforms, smart helmets, etc. For example, at least some of the sensing devices 500 may be implemented in a smart helmet configured to track a wearer's head motion and/or short-term and/or long-term impact of athletes' brain in different sports, such as (but not limited to) football, hockey, boxing, etc.

In some embodiments, the intelligent shoe is also configured to be paired with other smart wearables or devices, such as (but not limited to) smart shirts/uniforms, smart helmets, and/or smartwatches. For example, the data gathered from the intelligent shoes and the data gathered from a smart helmet of a same wearer can be integrated together to provide additional and/or more accurate insights related to pro-athlete performance and/or biometric.

In some embodiments, the data gathered from the intelligent shoes and/or other paired smart variables can further be analyzed and used in other industries as appropriate, such as (but not limited to) medical diagnosis, rescue services, and city planning/surveying. For example, in some embodiments, the intelligent shoes are configured to detect a fall of a wearer. When it is determined that a wearer had fallen and/or not moved for a predetermined time period, a notification may be sent to a family member, a nearby intelligent shoe, and/or a rescue service.

Further, the data gathered by the intelligent shoes, including the heatmap(s), can be used by the city or the governing body to create and/or revise safer paths for the visually impaired especially. Such data can also be used to design accessible housing and/or indoor navigation as well. For example, the paths that are frequently traveled should be maintained more frequently or be paved with tactile paving. In some embodiments, the paths that include tactile paving may be specially indicated in the map, and the intelligent shoes may be programmed to instruct visually impaired individuals to use those paths. Alternatively and/or additionally, an area that multiple people had fallen might indicate that the area is unsafe. In some embodiments, the unsafe area may be marked on the heatmap or map, such that the intelligent shoes will steer the wearers away from the unsafe area or generate an alert when the wearer is approaching the unsafe area. Alternatively, the organization (e.g., a city) that is responsible for maintaining the area may be notified to make safe of such areas or barricade/block the area.

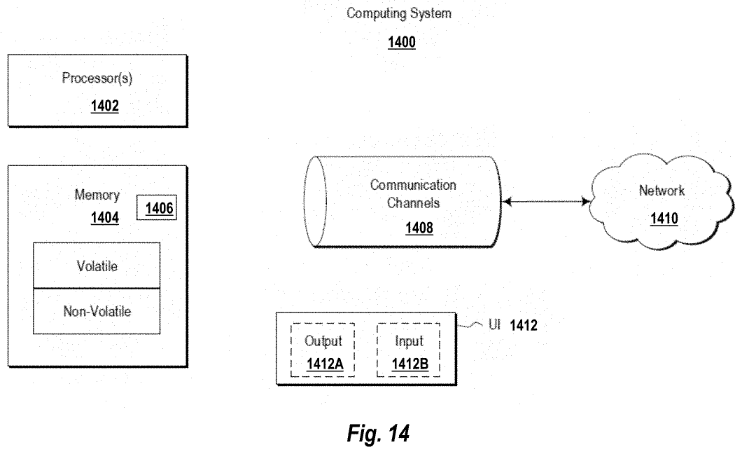

Finally, because the principles described herein may be performed in the context of a computing system (for example, each of intelligent shoe 110, 150, mobile device 120, 800 and server computing system 140, 700 may include one or more computing systems) some introductory discussion of a computing system will be described with respect to FIG. 14.

Computing systems are now increasingly taking a wide variety of forms. Computing systems may, for example, be handheld devices, appliances, laptop computers, desktop computers, mainframes, distributed computing systems, data centers, or even devices that have not conventionally been considered a computing system, such as wearables (e.g., glasses). In this description and in the claims, the term “computing system” is defined broadly as including any device or system (or a combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 14, in its most basic configuration, a computing system 1400 typically includes at least one hardware processing unit 1402 and memory 1404. The processing unit 1402 may include a general-purpose processor and may also include a field-programmable gate array (FPGA), an application-specific integrated circuit (ASIC), or any other specialized circuit. The memory 1404 may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well.

The computing system 1400 also has thereon multiple structures often referred to as an “executable component.” For instance, memory 1404 of the computing system 1400 is illustrated as including executable component 1406. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such a structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component.”

The term “executable component” is also well understood by one of ordinary skill as including structures, such as hardcoded or hardwired logic gates, that are implemented exclusively or near-exclusively in hardware, such as within a field-programmable gate array (FPGA), an application-specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “agent”, “manager”, “service”, “engine”, “module”, “virtual machine,” or the like may also be used. As used in this description and in the case, these terms (whether expressed with or without a modifying clause) are also intended to be synonymous with the term “executable component”, and thus also have a structure that is well understood by those of ordinary skill in the art of computing.

In the description above, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied in one or more computer-readable media that form a computer program product. An example of such an operation involves the manipulation of data. If such acts are implemented exclusively or near-exclusively in hardware, such as within an FPGA or an ASIC, the computer-executable instructions may be hardcoded or hardwired logic gates. The computer-executable instructions (and the manipulated data) may be stored in the memory 1404 of the computing system 1400. Computing system 1400 may also contain communication channels 1408 that allow the computing system 1400 to communicate with other computing systems over, for example, network 1410.

While not all computing systems require a user interface, in some embodiments, the computing system 1400 includes a user interface system 1412 for use in interfacing with a wearer. The user interface system 1412 may include output mechanisms 1412A as well as input mechanisms 1412B. The principles described herein are not limited to the precise output mechanisms 1412A or input mechanisms 1412B as such will depend on the nature of the device. However, output mechanisms 1412A might include, for instance, speakers, displays, tactile output, holograms, and so forth. Examples of input mechanisms 1412B might include, for instance, microphones, touchscreens, holograms, cameras, keyboards, mouse or other pointer input, sensors of any type, and so forth.

Embodiments described herein may comprise or utilize a special purpose or general-purpose computing system, including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general-purpose or special-purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM, or other optical disk storage, magnetic disk storage, or other magnetic storage devices, or any other physical and tangible storage medium which can be used to store desired program code means in the form of computer-executable instructions or data structures and which can be accessed by a general-purpose or special-purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general-purpose or special-purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RANI within a network interface module (e.g., a “NIC”), and then eventually transferred to computing system RANI and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general-purpose computing system, special purpose computing system, or special purpose processing device to perform a certain function or group of functions. Alternatively or in addition, the computer-executable instructions may configure the computing system to perform a certain function or group of functions. The computer-executable instructions may be, for example, binaries or even instructions that undergo some translation (such as compilation) before direct execution by the processors, such as intermediate format instructions such as assembly language, or even source code.

Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computing system configurations, including personal computers, desktop computers, laptop computers, message processors, handheld devices, multi-processor systems, microprocessor-based or programmable consumer electronics, network PCs, minicomputers, mainframe computers, mobile telephones, PDAs, pagers, routers, switches, data centers, wearables (such as glasses) and the like. The invention may also be practiced in distributed system environments where local and remote computing systems, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

The remaining figures may discuss various computing system which may correspond to the computing system 1400 previously described. The computing systems of the remaining figures include various components or functional blocks that may implement the various embodiments disclosed herein, as will be explained. The various components or functional blocks may be implemented on a local computing system or may be implemented on a distributed computing system that includes elements resident in the cloud or that implement aspect of cloud computing. The various components or functional blocks may be implemented as software, hardware, or a combination of software and hardware. The computing systems of the remaining figures may include more or less than the components illustrated in the figures, and some of the components may be combined as circumstances warrant. Although not necessarily illustrated, the various components of the computing systems may access and/or utilize a processor and memory, such as processing unit 1402 and memory 1404, as needed to perform their various functions.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An intelligent shoe, comprising:
one or more processors;
one or more sensorial devices;
a power source configured to power the one or more processors and the one or more sensorial devices; and
one or more computer-readable hardware storage device having stored thereon computer-executable instructions that are structured such that, when executed by the one or more processors, cause the intelligent shoe to perform at least:
cause a current location of a wearer of the intelligent shoe to be determined;
obtain a pedestrian path between the current location and a destination location; and
cause the one or more sensorial devices to generate sensorial signals to instruct the wearer of the intelligent shoe to walk along the pedestrian path from the current location to the destination location.

2. The intelligent shoe of claim 1, wherein the one or more sensorial devices are configured to instruct the wearer to move forward, stop, turn around, turn left, or turn right.

3. The intelligent shoe of claim 1, wherein the one or more sensorial devices comprise at least one of (1) an LED light, (2) a vibration device, (3) a color-changing fabric, (4) a speaker, or (5) a shape-changing fabric.

4. The intelligent shoe of claim 1, wherein a server is configured to generate the pedestrian path between the current location and the destination location, and the intelligent shoe is configured to obtain the pedestrian path generated by the server.

5. The intelligent shoe of claim 4, the intelligent shoe further configured to:
pair with a mobile device configured to communicate with the server; and
receive the pedestrian path generated by the server via the mobile device.

6. The intelligent shoe of claim 1, wherein the intelligent shoe further comprises one or more sensing devices;
the one or more sensing devices include at least one of:
a piece of smart fabric having an array of at least one of (1) one or more capacitive sensors, (2) one or more resistive sensors, (3) one or more conductive sensors, or (4) one or more NFC sensors;
a biometric sensor configured to securely authenticate a wearer;
a global positioning system (GPS) configured to obtain geolocation data of the intelligent shoe;
an inertial measurement unit (IMU) configured to measure motions of the intelligent shoe;
one or more body sensors configured to detect body conditions of the wearer; or
one or more environmental sensors configured to detect environmental conditions.

7. The intelligent shoe of claim 6, the intelligent shoe further configured to record sensing data generated by the one or more sensing devices.

8. The intelligent shoe of claim 7, the intelligent shoe further configured to detect abnormalities of gait based on the recorded sensing data.

9. The intelligent shoe of claim 7, the intelligent shoe further configured to record geolocation data associated with the intelligent shoe in the one or more computer-readable hardware storage device; and send the recorded geolocation data to a server.

10. The intelligent shoe of claim 1, wherein the power source includes:
a kinetic to electrical energy converter configured to convert kinetic energy of wearer of the intelligent shoe to electrical energy; and
a rechargeable battery configured to receive and store the electrical energy generated by the kinetic to electrical energy converter.

11. The intelligent shoe of claim 1, wherein the power source includes:
a solar panel configured to convert ambient light to electrical energy; and
a rechargeable battery configured to receive and store the electrical energy generated by the solar panel.

12. A server computing system, comprising:
one or more processors; and
one or more computer-readable media having stored thereon computer-executable instructions that are structured such that, when executed by the one or more processors, configure the server computing system to perform at least:
receive geolocation data generated from a plurality of intelligent shoes;
generate a heatmap indicating frequencies of visits of different areas based on the received geolocation data;
receive a navigation request containing at least a current location of a wearer of an intelligent shoe among the plurality of intelligent shoes;
in response to receiving the navigation request, generate a pedestrian path from the current location to a destination location based on the heatmap; and
send the pedestrian path to the intelligent shoe, causing the intelligent shoe to generate sensorial signals to instruct the wearer to walk along the pedestrian path.

13. The server computing system of claim 12, wherein the navigation request further includes a destination location, and the pedestrian path is generated based on areas between the current location and the destination location on the heatmap.

14. The server computing system of claim 12, wherein the pedestrian path is generated based on most or least traveled areas on the heatmap.

15. The server computing system of claim 12, wherein generating the pedestrian path includes generating a random pedestrian path based on the navigation request.

16. The server computing system of claim 15, wherein the navigation request includes one or more attributes of destination location, and the destination location is identified based on the one or more attributes.

17. The server computing system of claim 12, wherein the server computing system is further configured to accumulate environmental data associated with road conditions from the plurality of intelligent shoes and generate a second heatmap associated with road conditions, and the pedestrian path is also determined based on the second heatmap.

18. The server computing system of claim 12, wherein the server computing system is further configured to accumulate personal data associated with at least one wearer of the plurality of intelligent shoes, the personal data including data associated with gait of at least one wearer.

19. The server computing system of claim 18, the server computing system further configured to analyze the data associated with gait of the at least one wearer to identify abnormalities of the gait of the wearer.

20. A mobile device, comprising:
one or more processors; and
one or more computer-readable media having stored thereon computer-executable instructions that are structured such that, when executed by the one or more processors, configure the mobile device to perform at least:
pair with an intelligent shoe having one or more sensorial devices;
determine a current location of the mobile device;
receive a user input, indicating a navigation request;
obtain a pedestrian path from the current location to a destination location associated with the navigation request; and
send the pedestrian path to the intelligent shoe, causing the one or more sensorial devices of the intelligent shoe to generate sensorial signals to instruct a wearer of the intelligent shoe to walk along the pedestrian path.
